(19) United States
(12) Patent Application Publication
(10) Pub. No.: US 2001/0033841 A1
(43) Pub. Date: Oct. 25, 2001

(54) Modulating platelet function

(76) Inventors: Andrew D. Luster, Wellesley, MA (US); Sylvie Abi-Younes, Westwood, MA (US)

Correspondence Address: CLARK & ELBING LLP, 176 FEDERAL STREET, BOSTON, MA, 02110-2214, US

(21) Appl. No.: 09/804,606

(22) Filed: Mar. 12, 2001

Related U.S. Application Data
(63) Continuation of application No. 09/336,414, filed on Jun. 18, 1999, now abandoned.
(60) Provisional application No. 60/089,970, filed on Jun. 19, 1998.

(51) Int. Cl. A61K 39/395

(52) U.S. Cl. 424/146.1; 424/130.1

(57) ABSTRACT

Disclosed herein is a method of identifying a compound which affects the interaction between SDF-1 and platelets, comprising the steps of contacting SDF-1 with platelets in the presence of a test compound in a test sample;

(b) contacting SDF-1 with platelets in the absence of a test compound in a control sample;

(c) measuring the SDF-1 effect in said test and said control samples; and

(d) identifying compounds which increase or decrease said SDF-1 effect in the test sample compared to the control sample.

method of treating a patient with a vascular disease by administering an inhibitor of the interaction between stromal cell derived factor-1 (SDF-1) and platelets, in an amount effective to reduce the symptoms of said disease. Also disclosed is a method of stimulating the interaction between SDF-1 and platelets, as well as methods to identify compounds that modulate the above interaction.

### CROSS REFERENCE TO RELATED APPLICATIONS

This application is a continuation of and claims priority from U.S. patent application Ser. No. 09/336,414, filed on Jun. 18, 1999, which in turn claims priority from U.S. provisional application 60/089,970, filed on Jun. 19, 1998.

### BACKGROUND OF THE INVENTION

Platelets are circulating cytoplasmic megakaryocyte fragments that adhere to damaged vessels and aggregate to form a platelet plug, a process that is essential for hemostasis. However, in pathological states, the formation of acute platelet thrombi lead to vasoocclusion and ischemic necrosis, as, for example, in myocardial infarction and stroke. Coronary thrombosis, the immediate cause of acute coronary syndromes, usually results from atherosclerotic plaque disruption and in situ platelet aggregation (Murray et al., Lancet 349: 1498-1504, 1997; Libby, Circulation 91: 2844-50, 1995; Davies, Circulation 94: 2013-20, 1996; Ross, N. Engl. J. Med. 340: 115-26, 1999). Plaque rupture or erosion is associated with vascular endothelium damage, which changes the normally anti-thrombotic vessel into a prothrombotic surface, partly through exposure of subendothelial structures and perhaps also due to a local decrease in the production of platelet antagonists, such as endothelial-derived nitric oxide and prostacyclin (Ware et al., N. Engl. J. Med. 328: 628-35, 1996; Abrams, Am. J. Cardiol. 79: 2-9, 1997).

Chemokines are chemotactic cytokines that activate and direct the migration of leukocytes (Luster et al., N. Engl. J. Med. 388: 436-45, 1998; Rollins, Blood 90: 909-28, 1997). They are produced from multiple cells, including endothelial cells and macrophages during vessel injury and atherosclerosis. Chemokines may also have other roles beyond leukocyte chemotaxis. For example, mice deficient in the chemokine stromal derived factor-1 (SDF-1) have defects in B-cell lymphopoiesis and bone marrow myelopoiesis and die perinatally with cerebellar, cardiac, and vascular morphogenic abnormalities (Nagasawa et al., Nature 382: 635-38, 1996; Tachibana et al., Nature 393: 591-94, 1998; Zou et al., Nature 393: 595-99, 1998; Ma et al., Proc. Natl. Acad. Sci. USA 95: 9448-53, 1998). The gene encoding the chemokine SDF-1 can be alternatively spliced to produce SDF-1α or SDF-1β; SDF-1β contains an additional 3′ exon encoding four C-terminal amino acids (Tashiro et al., Science 261: 600-03, 1993; Shirozu et al., Genomics 28: 495-500, 1995).

### SUMMARY OF THE INVENTION

The invention provides a method of treating a patient with atherosclerosis, involving administering to said patient an inhibitor of SDF-1 or CXCR4 biological activity, said administering in an amount effective to reduce said symptoms of said atherosclerosis, said inhibitor being an inhibitor of T-cell or monocyte chemotaxis. The invention also features a method identifying a compound which affects the interaction between SDF-1 and platelets involving the steps of (a) contacting SDF-1 with platelets in the presence of a test compound in a test sample, (b) contacting SDF-1 with platelets in the absence of a test compound in a control sample, (c) measuring the SDF-1 effect (e.g., platelet aggregation and calcium flux) in the test and the control samples, and (d) identifying compounds which increase or decrease the SDF-1 effect in the test sample compared to the control sample.

The invention also includes a method of inducing platelet activation involving stimulating the interaction between SDF-1 and platelets (e.g., the interaction between SDF-1 and the platelet chemokine receptor, CXCR4). Preferably, the interaction is stimulated by administering SDF-1.

Also included is a method of treating a patient with decreased platelet number or function, the method involving stimulating the interaction between SDF-1 and platelets. Preferably, the interaction is stimulated by administering SDF-1 to the patient in an amount effective to reduce the symptoms of the disease.

The invention also features a method of identifying a patient at risk of developing acute thrombosis or atherosclerosis involving measuring SDF-1 level or SDF-1 activity in the patient's blood, wherein increased SDF-1 level or activity indicates the increased risk.

In another aspect, the invention features a method of treating a patient with a vascular disease (e.g., atherosclerosis, acute thrombosis, the method involving administering to the patient an inhibitor of the interaction between stromal cell derived factor-1 (SDF-1) and platelets, in an amount effective to reduce the symptoms of the disease. Preferably, the treatment reduces the occurrence of stroke, myocardial infarction, pulmonary embolism, or deep vein thrombosis, reduces the disruption of atherosclerotic plaques, reduces the platelet-induced thrombus formation. In another preferred embodiment, inhibitor inhibits the interaction between SDF-1 and the platelet chemokine receptor, CXCR4. Preferably, the inhibitor is an antibody to SDF-1, an antibody to CXCR4, or a CXCR4 inhibitor (e.g., T22[Tyr^{5,12}, Lys⁷]-polyphemusin II, ALX40-4C, or AMD3100).

By “SDF-1 biological activity” is meant those activities of the polypeptide which naturally occur in vivo or in vitro, these activities include effects on monocyte chemotaxis.

By “CXCR4 biological activity” is meant those activities of the receptor which naturally occur in vivo or in vitro, these activities include effects on monocyte chemotaxis

By “vascular disease” is meant a condition in which the cells lining a blood vessel experience an inflammatory response, or are infiltrated by exogenous cells, or proliferate, or undergo plaque formation, in such a manner that the cross-sectional area of the lumen of the blood vessel is reduced, as compared to a normal vessel.

By “interaction between SDF-1 and platelets” is meant a communication between the cytokine and the cells, for example, a communication by means of SDF-1 binding to a platelet CXCR4 receptor, which induces a detectable response in the platelets. Such detectable cellular responses include platelet aggregation, increased cytosolic calcium, activation of phosphatidyl inositol-3 kinase, activation of tyrosine kinases, or activation of cyclooxygenase.

### BRIEF DESCRIPTION OF THE DRAWING

FIGS. 1A-1C demonstrate SDF-1 induced platelet aggregation. FIG. 1A is a representative tracing illustrating the aggregatory effect of 40 nM SDF-1α compared to the same dose of 15 chemokines, 6 CXC chemokines (IP-10, NAP-2, IL-8, ENA-78, GROa, and MIG) and 9 CC chemokines (MCP-1, MCP-2, MCP-3, MCP-4, eotaxin, RANTES, MIP-1α, MIP-1β, and I-309) (2 donors, n=2). FIG. 1B is a representative tracing of platelets stimulated with increasing concentrations of SDF-1α (10 donors, 20 experiments, n=91). FIG. 1C is a representative tracing of platelets stimulated with increasing concentrations of SDF-1β (2 donors, 3 experiments, n=12). Open arrow head indicates the primary aggregatory response and solid arrow head indicates the secondary response.

FIG. 2A and 2B show representative tracings of SDF-1 induced calcium flux in platelets from 2 individual donors. Fura-2 loaded platelets were stimulated with 100 nM SDF-1α at the time indicated by the arrow. Intracellular calcium levels were expressed as the ratio of fluorescence excitation at 340/380 nM over time (3 donors, n=6). In FIG. 2B, thrombin (1U/ml) was used as a positive control at the time indicated by the arrow.

FIG. 3 demonstrates that platelets express CXCR4, the SDF-1 receptor. Flow cytometry analysis was conducted on platelets using a CXCR4 monoclonal antibody (MAB 173) (solid line) and the isotype matched control monoclonal antibody (dotted line) (n=2).

FIG. 4A and 4B illustrate the signaling pathway of SDF-1 mediated platelet aggregation. FIG. 4A illustrates that SDF-1 induced platelet aggregation is mediated via the CXCR4 receptor. Anti-CXCR4 monoclonal antibody (12G5) blocked SDF-1 mediated platelet aggregation. Platelets were incubated for 1 minute with anti-CXCR4 mAb (10 μg/ml), or an isotype matched control mAb prior to the addition of SDF-1α 80 ng/ml (2 donors, n=3). FIG. 4B illustrates that the aggregatory effect of SDF-1α (80 ng/ml) on platelets was blocked by a 30 minute preincubation with pertussis toxin (1.7 nM, 2 donors, n=2).

FIG. 5A shows the inhibitory effect of aspirin (1 mM, 2 donors, n=2). FIG. 5B shows the inhibitory effect of genestein (200 μM, 3 donors, n=3). Platelets were incubated with genestein or vehicle for 5 minutes before addition of SDF-1. FIG. 5C shows the inhibitory effect of wortmannin (20 μM, 3 donors, n=3).

FIG. 6 illustrates that SDF-1 protein is expressed in human atherosclerotic plaques. Western blot analysis of 4 carotid artery plaques and 3 normal arteries all isolated from different individuals showing increased expression of SDF-1 in atherosclerotic plaques compared to normal vessels. rSDF-1 is recombinant human SDF-1α. The arrow indicates the position of SDF-1. The molecular weight in kDa is indicated on the left of the blot.

FIG. 7A and 7B demonstrate that SDF-1 is expressed in various cell types in human atheroma. FIG. 7A shows immunoperoxidase staining of SDF-1 in a normal carotid artery and in an atherosclerotic plaque using a goat anti-SDF-1 polyclonal antibody. As a control, an adjacent section of the atherosclerotic plaque was stained with a non-immune goat IgG. SDF-1 was not detected in the normal vessel but was detected in the plaque, while the control IgG did not stain the plaque. (100× magnification). FIG. 7B shows the colocalization of SDF-1 in CD31+ endothelial cells (EC), α-actin+ smooth muscle cells (SMC), and CD68+ macrophages (MO) in a representative plaque. The arrow indicates endothelial cells stained for SDF-1 (400× magnification).

### DETAILED DESCRIPTION OF THE INVENTION

Our studies indicate a role for SDF-1 in platelet-associated hemostasis and in the pathogenesis of atherogenic and thromboocclusive diseases. As further discussed below, we studied the direct effect of sixteen chemokines on human platelets and found that only SDF-1 induced a platelet aggregatory effect. Furthermore, this effect was inhibited by both pertussis toxin and an antibody to the chemokine receptor, CXCR4. Since atherosclerotic vessels are prone to develop platelet-rich thrombi, we examined the expression of SDF-1 in human atheroma. SDF-1 protein was highly expressed in smooth muscle cells, endothelial cells and macrophages in human atherosclerotic plaques but not in normal vessels.

The invention features a method for inhibiting thrombosis and platelet aggregation and a method for stabilizing atherogenic plaques in a patient in need thereof. Preferred patients include, but are not limited to, those developing, or at risk of developing, thrombosis, atherosclerosis, stroke, myocardial infarction, pulmonary embolism, or deep vein thrombosis, as well as patients with disorders associated with increased SDF-1 expression or activity. The method includes administration of a therapeutically effective dose of a compound which inhibits the interaction between SDF-1 and platelets, for example, by inhibiting the interaction between SDF-1 and the platelet CXCR4 receptor.

Molecules which inhibit the interaction of SDF-1 and CXCR4 include antibodies which bind either of the proteins (e.g., CXCR4-specific mAb, 12G5, D'Apuzzo et al., Eur. J. Immunol. 27:1788-1793 (1997); Bleul et al., Proc. Natl. Acad. Sci. USA 94:1925-1930 (1997)), peptides (e.g., T22[Tyr^{5,12},Lys⁷]-polyphemusin II, a synthesized peptide that consists of 18 amino acid residues, and is an analogue of polyphemusin II, isolated from the hemocyte debris of American horseshoe crabs (Limulus polyphemus), Murakami et al., J. Exp. Med. 186(8) 1389-1393 (1997), and other T22-derived analogues; and ALX40-4C, a small peptide of nine Arg residues stabilized by terminal protection and the inclusion of D amino acids, Doranz et al., J. Exp. Med. 186(8):1395-1400 (1997)), and small synthetic molecules (e.g., bicyclams, such as AMD3100, previously called JM3100 or SID791, Schols et al., J. Exp. Med. 186(8):1383-1388 (1997)). Inhibitors of the SDF-1/platelet interaction, specifically formulated for administration to a patient at risk for or undergoing thrombosis, platelet aggregation, or in need of plaque stabilization, are also a feature of the invention.

In addition, the invention features a method for inducing platelet aggregation in a patient in need thereof, for example, in patients with bleeding diathesis, thrombocytopenia, or related platelet disfunction. Preferred patients include those with platelet insufficiency which may or may not be associated with decreased SDF-1 levels or decreased SDF-1 activity. The method includes administration of a therapeutically effective dose of a compound which stimulates the interaction between SDF-1 and platelets, for example, the interaction between SDF-1 and the platelet CXCR4 receptor. Such administration may involve delivery via local SDF-1 deposition such as local SDF-1 injection or SDF-1 containing implant. Molecules which stimulate the interaction of SDF-1 and CXCR4 include SDF-1 itself, as well as SDF-1 related peptides (Heveker et al., Curr. Biol. 8: 369-76, 1998; Crump et al., EMBO J. 16: 6996-7007, 1997). Agonists of the SDF-1 platelet interaction, specifically formulated for administration to a patient in need of platelet aggregation, are also a feature of the invention.

A related feature of the invention is a method of screening patients that are developing, or are at increased risk of developing, atherogenesis, thromboocclusion, or platelet insufficiency, to determine if defects in SDF-1 expression or activity, or antibody-mediated alteration of the interaction between SDF-1 and CXCR4, play a role in the pathogenesis of these diseases. This method involves screening the patients for mutations in the SDF-1 gene, SDF-1 protein levels, or SDF-1 activity, and has the advantage of identifying patients with abnormally high or low SDF-1 expression or activity. If a patient's SDF-1 expression or activity is abnormally high, the patient is likely to benefit from treatment that inhibits the SDF-1/platelet interaction. If, on the other hand, a patient's SDF-1 expression or activity is abnormally low, the patient is likely to benefit from treatment that enhances the SDF-1/platelet interaction. In addition, SDF-1 treatment can, in some instances, be beneficial to increase SDF-1 levels regardless of the patient's baseline level

The invention also provides methods for identifying additional compounds that modify the interaction between SDF-1 and platelets. These methods are discussed in further detail below. Such compounds will be useful therapeutically, to inhibit or enhance the interaction between SDF-1 and platelets, as needed. The method includes assaying compounds for their effect on SDF-1/platelet interaction, as determined by, for example, SDF-1 binding to platelets, calcium flux into platelets, and platelet aggregation. This invention also features a method of identifying SDF-1 homologues that bind the CXCR4 receptor, but do not induce platelet aggregation. Such compounds are useful to administer, for example, to prevent the intracellular transfer of human immunodeficiency virus (HIV) without causing the potential adverse side effect of platelet aggregation which could result from SDF-1 treatment.

### EXAMPLE 1

We studied the response of human platelets to 16 chemokines [stromal derived factor-1 α and β (SDF-1α and SDF-1β), interferon-inducible protein of 10 kD (IP-10), neutrophil-activating peptide 2 (NAP-2), interleukin-8 (IL-8), epithelial cell derived neutrophil-activating protein (ENA-78), growth-regulated oncogene-a (GROa, monokine induced by interferon-g (MIG), monocyte chemoattractant protein-1 (MCP-1), MCP-2, MCP-3 and MCP-4, eotaxin, regulated on activation normal T-cell expressed and secreted (RANTES), macrophage inflammatory protein-1a (MIP-1a) and MIP-1b)] and I-309.

1. Platelet Aggregation

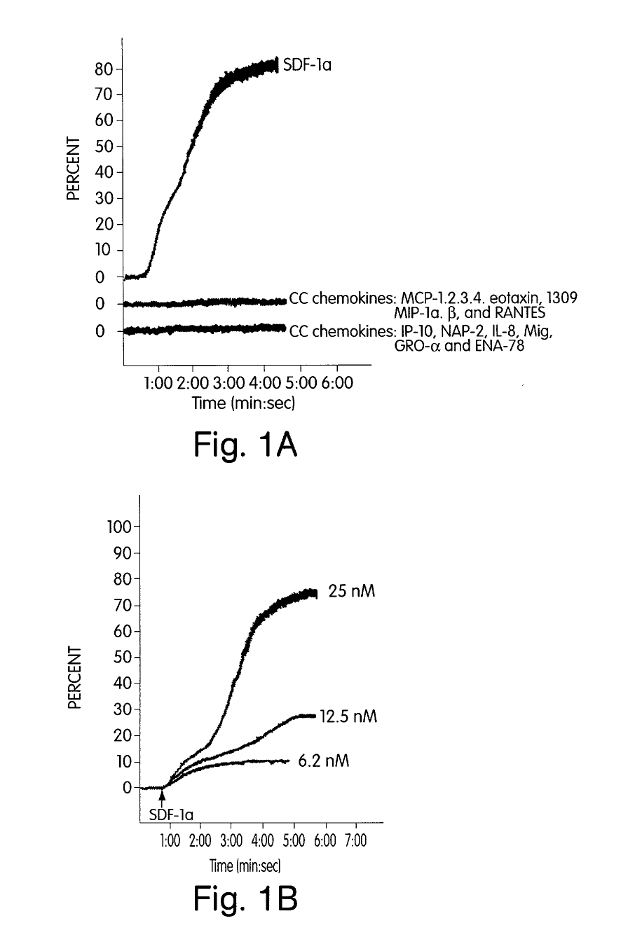

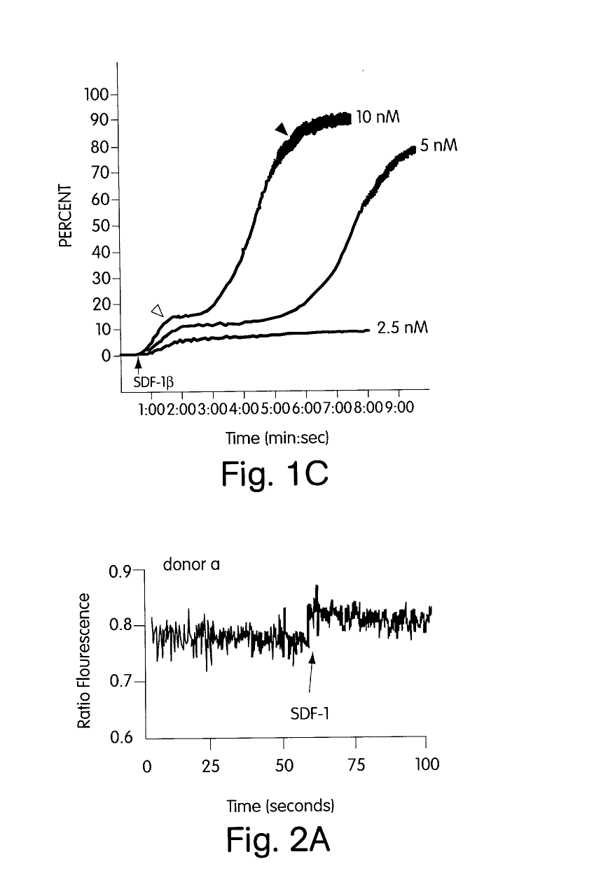

Of the chemokines tested, only SDF-1 induced platelet aggregation (FIG. 1A). The SDF-1α and β effects on platelets were concentration dependent (FIG. 1B and 1C, respectively). The concentration of SDF-1α and SDF-1β necessary to induce a maximum aggregatory response ranged between 10 and 100 nM.

Platelets have several levels of response to stimuli. The first level consists of platelet shape change seen as a minor change in aggregometer traces. Primary aggregation is the second level of response, defined as aggregation without secretion and is at least partially reversible. Secondary aggregation, the third level of activation, is associated with maximal irreversible aggregation, platelet granule secretion and prostanoid synthesis. Low concentrations of SDF-1 only induced the primary phase of aggregation (6.2 and 2.5 nM for SDF-1α and 1β, respectively, FIGS. 1B and 1c). However, with increasing amounts of either SDF-1α (12.5-25 nM, FIG. 1B) or 1β (5-10 nM, FIG. 1C), a primary and secondary response was observed (see, for example, the open and closed arrow, respectively, FIG. 1C). Out of twelve donors tested, ten had a fall primary and secondary aggregation response to SDF-1, while the remaining two responded with only primary aggregation. These data suggest that other factors also regulate platelet responsiveness to SDF-1 stimulation.

Given that SDF-1 is chemotactic for resting T-cells, monocytes, B cell precursors, natural killer cells and CD34+ stem cells, with maximal chemotaxis seen with 10-100 nM SDF-1 (Bleul et al., J. Exp. Med. 184:1101-09, 1996; Campbell et al., Science 279: 381-84, 1998), the SDF-1 dose range necessary to achieve a maximal aggregatory effect on platelets is comparable to that required for a maximal chemotactic effect.

2. SDF-1 Induced Calcium Flux

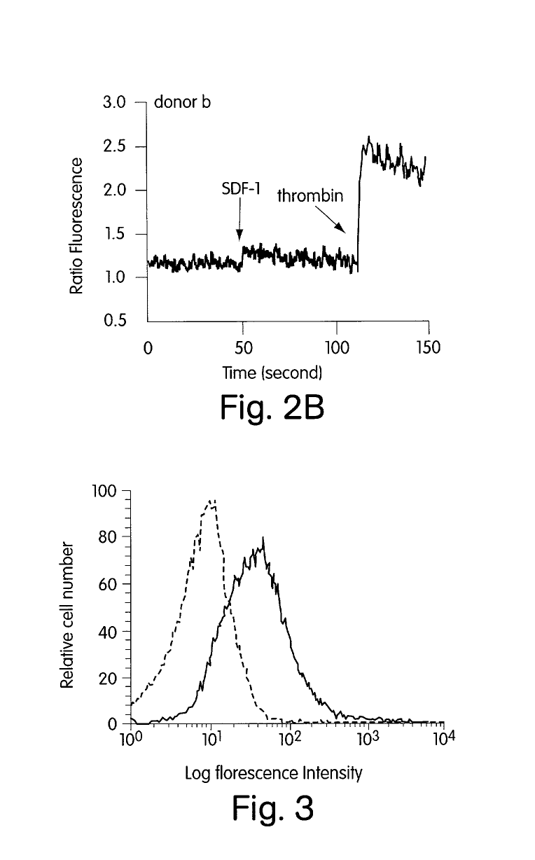

In human platelets, SDF-1 (50-100 nM) induced an increase in cytosolic calcium (FIG. 2A and 2B).

3. CXCR4 Expression and Signaling

SDF-1 signals cells through the CXC-chemokine receptor 4 (CXCR4), a seven transmembrane spanning G protein-coupled cell-surface glycoprotein. We found that human peripheral blood platelets expressed CXCR4, as determined by flow cytometry, using two different monoclonal antibodies specific for CXCR4 (FIG. 3).

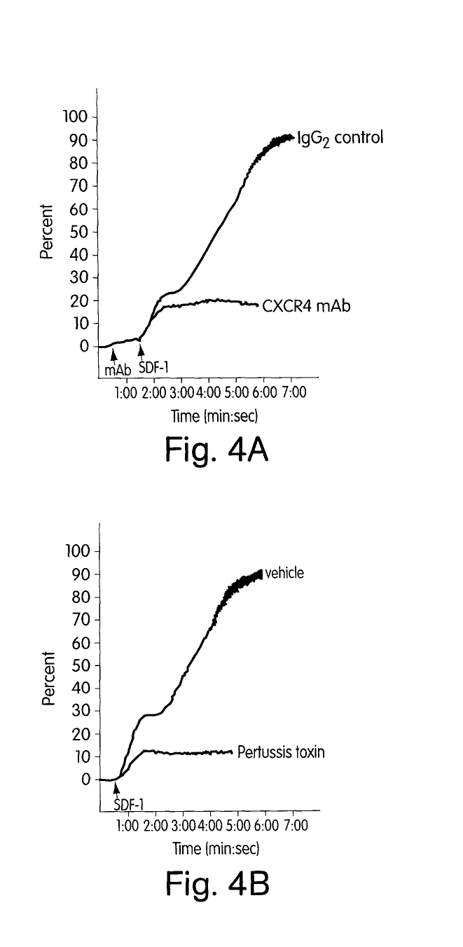

A monoclonal antibody to CXCR4 inhibited SDF-1 induced platelet aggregation by more than 50%, confirming that SDF-1 signals platelets through CXCR4 (FIG. 4A). An isotype matched control antibody had no effect on SDF-1 induced platelet aggregation. SDF-1 induced platelet aggregation was also blocked by pertussis toxin (FIG. 4B), confirming that this effect was mediated, at least in part, by a pertussis toxin-sensitive G protein, such as Gαi. The partial SDF inhibition by pertussis toxin could result from the CXCR4 coupling to multiple G proteins, where at least one G protein is pertussis toxin sensitive, for example, Gαi, and another is pertussis toxin insensitive, for example, Gαq.

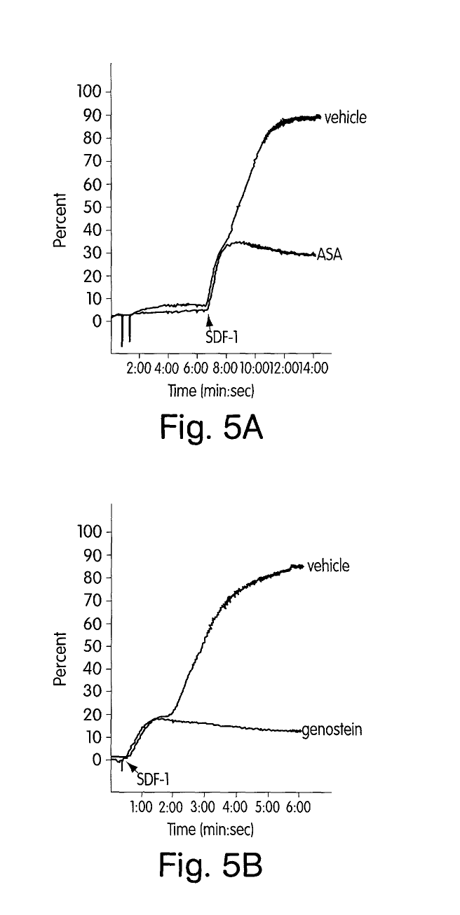

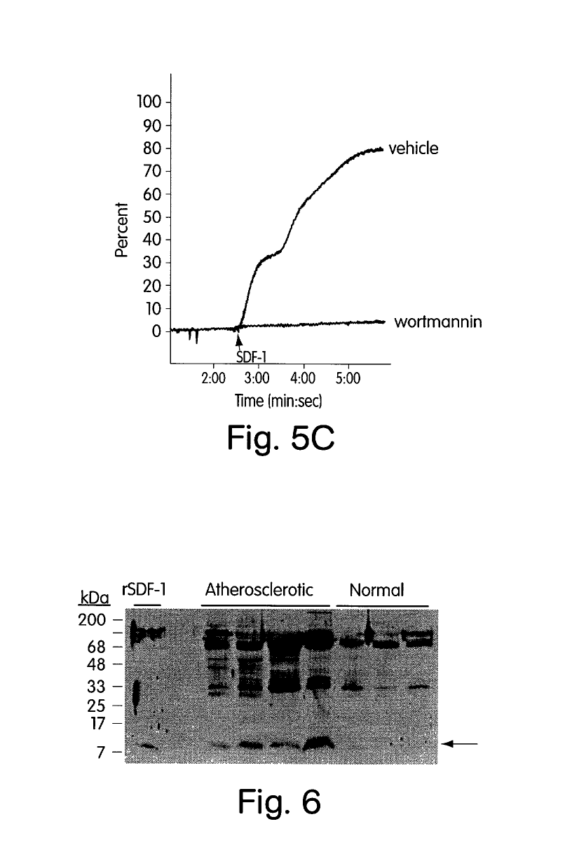

As shown in FIG. 5, SDF-1 induced platelet aggregation was studied in the presence of known modifiers of platelet function, for example, aspirin, genestein, and wortmannin (Ware et al., In Williams Hematology, Eds, Boulter et al., McGraw Hill, 1161-1200, 1995; Furman et al., Circ. Res. 75: 172-80, 1994; Furman et al., Proc. Natl. Acad. Sci. USA 95: 3082-87, 1998). Aspirin, a platelet cyclooxygenase inhibitor, inhibited SDF-1 induced secondary aggregation, indicating that prostanoid synthesis is required for SDF-1 induced secondary aggregation (FIG. 5A). Genestein, a tyrosine kinase inhibitor, also decreased SDF-1 platelet aggregation (FIG. 5B). In addition, wortmannin, an inhibitor of phosphatidyl inositol-3 kinase (PI-3 kinase) and, at higher concentrations, a myosin light chain kinase inhibitor, completely inhibited SDF-1 induced platelet aggregation (FIG. 5C). Without wishing to be bound by any particular theory, the above results suggest that SDF-1 induced platelet aggregation involves activation of PI-3 kinase and/or myosin light chain kinase, and depends, at least in part, on prostanoid synthesis and tyrosine kinase activity.

4. Atherosclerotic Plaques Express SDF-1

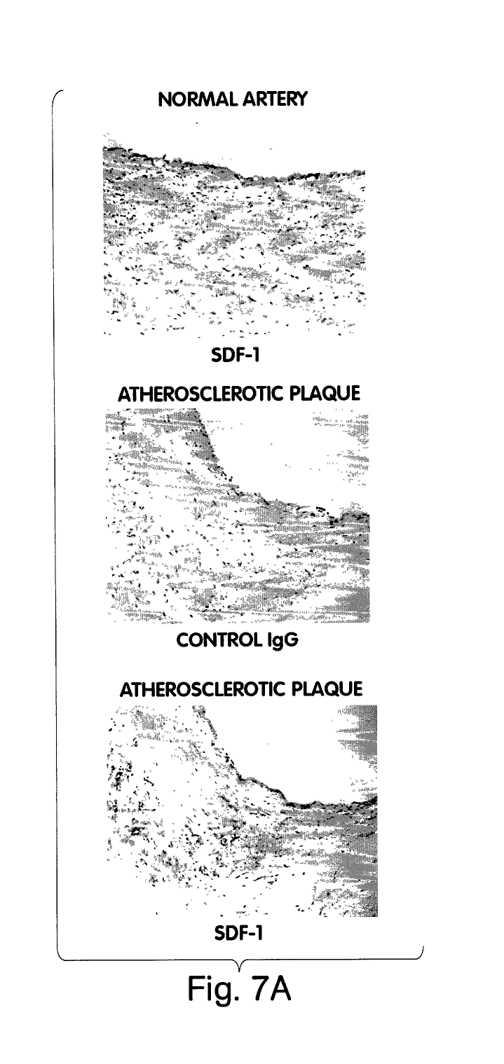

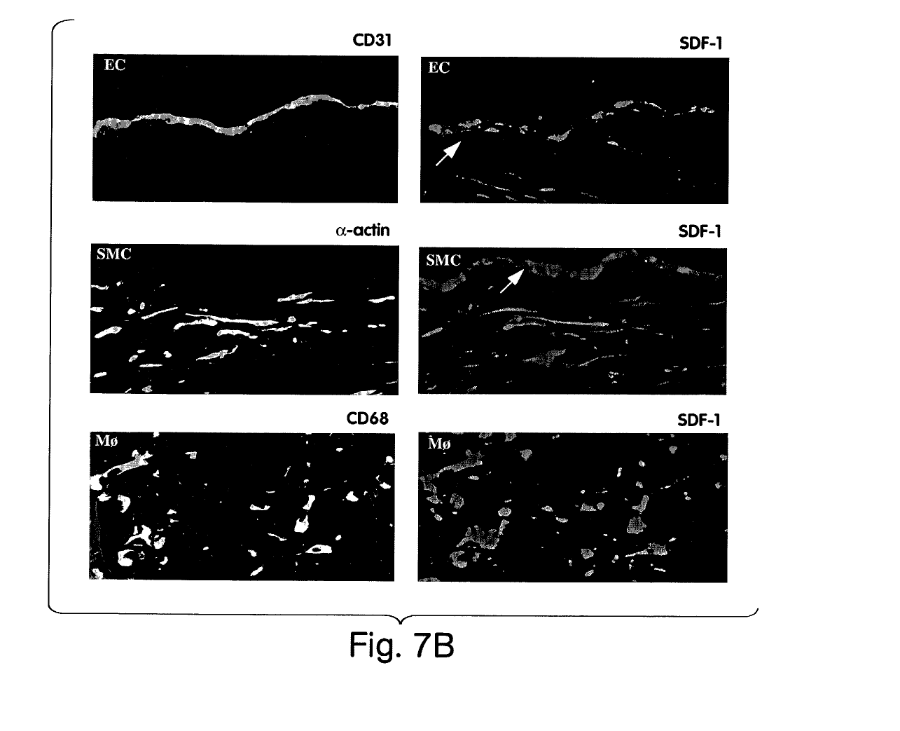

Considering that platelet activation is central to the pathogenesis of hemostasis and arterial thrombosis, we investigated the expression of SDF-1 protein in human atherosclerotic plaque lysates. Western blot analysis revealed a striking increase in SDF-1 immunoreactivity in atherosclerotic plaques isolated from four different carotid atheromas compared to non-atherosclerotic arteries (FIG. 6). Immunohistochemical staining, using two different anti-SDF-1 specific antibodies, showed abundant expression of SDF-1 protein in atheromatous arteries but not in normal arteries (FIG. 7A). Double immunoflourescence colocalized SDF-1 staining in the plaque to endothelial cells (CD31+), smooth muscle cells (α-actin+), and macrophages (CD68+) (FIG. 7B).

5. Methods

Blood collection and platelet preparation. Human blood was collected from antecubital veins of healthy male or female, aspirin-free volunteers into syringes containing heparin (10 units/ml final concentration) for flow cytometry studies or sodium citrate (0.38% final concentration) for aggregation studies. Platelet rich plasma (PRP) was prepared by centrifugation of whole blood at 150 g for 15 minutes. Platelet poor plasma (PPP) was prepared by centrifugation of PRP at 1200 g for 15 minutes.

Aggregation studies. Experiments were performed using a Chrono-Log model 560 vs or 490-2D aggregometer (Havertown, Pa.). Aliquots of PRP (0.50 ml or 0.45 ml) with a platelet concentration of 2-3×105 platelets/ml were incubated at 37° C. and stirred at 1000 rpm. Chemokines (PeproTech Inc., Rocky Hill, N.J., except SDF-1β, which was obtained from Genetics Institute, Boston, Mass.) were added and aggregation was measured as a percent change in optical density, with the instrument calibrated to yield 0% change in optical density for PRP and with the PPP 100% standard for change in optical density. Aggregation scale was set so that maximal aggregation gave 85-90% chart deflection. Inhibition experiments were done using CXCR4 mAb 12G5 (R&D, Minneapolis, Minn.), polyclonal anti-SDF-1 (R&D, Minneapolis, Minn.), pertussis toxin (Sigma, St. Louis, Mo.), wortmannin (Sigma), genestein (Sigma), and aspirin (Sigma). DMSO was used as vehicle for genestein and wortmannin and 1N NaOH was used as a vehicle for aspirin.

Flow cytometry. Platelets were analyzed by flow cytometry using fixed whole blood as previously described. Double staining was performed with mouse anti-human CXCR4 mAbs MAB 173 or 12G5 (R&D) followed by FITC-conjugated F(ab)2 goat anti-mouse IgG (ImmunoTech) and phycoerythrin-conjugated mouse anti-human CD41a (anti-glycoprotein IIb/IIIa) monoclonal antibody (Pharmingen, San Diego, Calif.).

Calcium flux. Fura-2 loaded platelets were prepared from acid citrate dextrose anti-coagulated blood (Rink et al., J. Physiol. 393: 513-24, 1987). PRP was collected by centrifugation for 15 minutes at 200 g and 100 μM aspirin was added. Platelets were then loaded with fura-2 by incubating PRP with 2 μM acetoxymethyl ester of fura-2 (fura-2 AM; Molecular Probes, Inc., Eugene, Oreg.) for 45 minutes at 37° C. in the dark. PRP was then centrifuged at 1500 g for 10 minutes and the pellet washed and resuspended in a buffer containing 145 mM NaCl, 4 mM KCl, 1 mM NaH₂PO₄, 0.8 mM MgCl₂, 1.8 mM CaCl₂, 25 mM Hepes and 22 mM glucose. Changes in cytosolic free calcium were determined after addition of SDF-1 (500 or 1000 ng/ml) by monitoring the excitation fluorescence intensity emitted at 510 nm in response to sequential excitation at 340 nm and 380 nm using a Delta RAM (Random Access Monochromator) fluorimeter (Photon Technology International, Monmouth Junction, N.J.). The data are presented as the relative ratio of fluorescence at 340/380 nm.

Western blotting. Surgical specimens of human carotid atheroma and aorta were homogenized in a mixture of 20 mM NaCl, 200 mM Tris-HCl (pH 7.6) and 10% SDS. Extracts were separated (200 mg proteins/lane) by standard SDS-PAGE under reducing conditions, and blotted on to polyvinylidene difluoride membranes (Bio-Rad, Hercules, Calif.) using a semi-dry blotting apparatus (0.8 mA/cm², 30 min; Bio-Rad). Blots were blocked and dilution of first and second antibody was made in 5% dry skim milk/PBS/0.1% Tween. After 1 hour of incubation with the primary goat anti-human SDF-1 antibody (R&D), blots were washed three times (PBS/0.1% Tween) and the secondary peroxidase-conjugated rabbit-anti-goat antibody (Jackson Immunoresearch, West Grove, Pa.) was added for another hour. Finally, after washing the blots, detection of the antigen was carried out using the enhanced chemiluminescent detection method (Dupont-NEN, Boston, Mass.).

Immunohistochemistry. Atherosclerotic plaques from human carotid arteries and non-atherosclerotic arteries were obtained from endarterectomy transplant donors and autopsies by protocols approved from the Human Investigation Review Committee at the Brigham and Women's Hospital. Serial cryostat sections (6 mm) were cut, air dried onto microscope slides (Fisher Scientific, Melverne, Pa.), and fixed in acetone at −20° C. for 5 minutes. Sections preincubated with PBS containing 0.3% hydrogen peroxidase activity were incubated (60 minutes) with the primary goat anti-human SDF-1 antibodies (R&D, and Santa Cruz Biotechnology) or control antibody, diluted in PBS supplemented with 5% appropriate serum. Finally, sections were incubated with the respective biotinylated secondary antibody (45 minutes, Vector Laboratories) followed by avidin-biotin-peroxidase complex (Vectastain ABC kit), and antibody binding was visualized with 3-amino-9-ethyl carbazole (Vector Laboratories). Cell types were characterized by double immunofluorescence staining using anti-muscle α-actin mAb specific for smooth muscle cells (Enzo Diagnostics, New York, N.Y.), anti-CD31 mAb specific for endothelial cells (Dako), anti-CD68 mAb specific for macrophages (Dako), using FITC (cell-specific antibody) and Texas-red (SDF-1α specific antibody) conjugated streptavidin.

6. SDF-1 Related Conditions and Therapies

The above illustrated SDF-1 expression pattern and platelet activation effects indicate that SDF-1 plays a role in platelet-rich thrombus formation following plaque disruption and in the pathogenesis of atherosclerosis. Given that a prothrombotic surface can have reduced platelet antagonists, such as endothelial-derived nitric oxide and prostacyclin, the SDF-1 effect would be enhanced, further inducing platelet activation, aggregation and platelet thrombus formation.

SDF-1 mediated platelet activation sets into motion further platelet action which contributes to the development of atherosclerosis, given that activated platelets release their own pro-inflammatory cytokines, chemokines, and lipid metabolites (Ross, Nature 362: 801-09, 1993; Kameyoshi et al., J. Exp. Med. 176: 587-92, 1992). In addition, activated platelets express the CD40 ligand and P-selectin which induce chemokine secretion from endothelial cells and monocytes, respectively (Weyrich et al., J. Lin. Invest. 97: 1525-34, 1996; Henn et al., Nature 391: 591-94, 1998).

In addition to its role as a platelet activator, SDF-1 is itself a potent chemotactic for T cells and monocytes and has been shown to arrest the flow of circulating lymphocytes (Bleul et al., J. Exp. Med. 184: 1101-09, 1996). T cells and monocytes are known to be involved in the pathogenesis of plaque rupture. Taken alone or together, the SDF-1 and platelet associated pathways described above serve to amplify an inflammatory response at the site of plaque rupture and increase thrombotic formation.

Our findings demonstrate a role for SDF-1 in the recruitment and subsequent retention of inflammatory cells in atherosclerotic vessels by stimulating platelets to aggregate and also to release their chemokines and cytokines. Therefore, inhibiting the interaction between SDF-1 and CXCR4 will provide therapeutic benefit by inhibiting vessel inflammation and platelet aggregation, thereby stabilizing atherosclerotic plaques and reducing thrombus formation, respectively.

The SDF-1 receptor, CXCR4, also functions as an entry cofactor for T-tropic HIV-1 by binding to the gp120 HIV coat protein. Our finding that CXCR4 is present on platelets and induces platelet aggregation suggests that CXCR4 may play a role in HIV-induced thrombocytopenia. SDF-1 is a powerful inhibitor of T-tropic HIV-1 infection and is being evaluated as a possible new therapy for HIV. This concept has gained momentum following the discovery that a polymorphism in the SDF-1 gene is associated with delayed progression of HIV disease. Our studies raise the concern that such SDF-1 therapy could result in increasesd platelet activation as a side effect. Identifying SDF-1 like compounds that block HIV entry without inducing platelet aggregation could overcome this problem.

Identification of Compounds that Modulate SDF-1 Activity and Platelet Aggregation

Modulating the interaction between SDF-1 and platelets (as demonstrated in the Example 1, supra), affects platelet-associated hemostasis, platelet aggregation, and atherosclerotic plaque disruption. This finding allows us to provide screening assays for drugs which modulate SDF-1 induced platelet activation. Such assays may measure SDF-1 induced platelet activation by measuring changes in: (a) in vitro and in vivo SDF-1 binding to CXCR4; (b) aggregation of platelets; (c) calcium flux and cytosolic calcium levels in platelets; and (d) levels of SDF-1 mRNA or gene expression. Such identified compounds may have therapeutic value in the treatment or prevention of diseases such as atherosclerosis, stroke, myocardial infarction, and SDF-1 associated platelet deficiency.

Test Compounds

In general, novel drugs for prevention or treatment of platelet-associated disorders, which function by targeting the SDF-1/platelet interaction are identified from large libraries of both natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their therapeutic activities for neurodegenerative disorders should be employed whenever possible.

When a crude extract is found to modulate the SDF-1 interaction with platelets, further fractionation of the positive lead extract is necessary to isolate chemical constituents responsible for the observed effect. Thus, the goal of the extraction, fractionation, and purification process is the careful characterization and identification of a chemical entity within the crude extract having effects on the SDF-1/platelet interaction. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art.

Therapy

Compounds identified using any of the methods disclosed herein, may be administered to patients or experimental animals with a pharmaceutically-acceptable diluent, carrier, or excipient, in unit dosage form. Conventional pharmaceutical practice may be employed to provide suitable formulations or compositions to administer such compositions to patients or experimental animals. Although intravenous administration is preferred, any appropriate route of administration may be employed, for example, parenteral, subcutaneous, intramuscular, intracranial, intraorbital, ophthalmic, intraventricular, intracapsular, intraspinal, intracistemal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for antagonists or agonists of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Other embodiments

References cited herein are hereby incorporated by reference.

What is claimed is:

1. A method of treating a patient with atherosclerosis, said method comprising administering to said patient an inhibitor of SDF-1 or CXCR4 biological activity, said administering in an amount effective to reduce said symptoms of said atherosclerosis, said inhibitor being an inhibitor of T-cell or monocyte chemotaxis.

2. A method of treating a patient with a vascular disease, said method comprising administering to said patient an inhibitor of the interaction between stromal cell derived factor-1 (SDF-1) and platelets, in an amount effective to reduce the symptoms of said disease.

3. The method of claim 2, wherein said vascular disease is atherosclerosis.

4. The method of claim 3, wherein said inhibitor reduces the disruption of atherosclerotic plaques.

5. The method of claim 2, wherein said vascular disease is acute thrombosis.

6. The method of claim 2, wherein said inhibitor reduces the platelet-induced thrombus formation.

7. The method of claim 2, wherein said inhibitor reduces the occurrence of stroke, myocardial infarction, pulmonary embolism, or deep vein thrombosis.

8. The method of claim 2, wherein said inhibitor inhibits the interaction between SDF-1 and the platelet chemokine receptor, CXCR4.

9. The method of claim 8, wherein said inhibitor is an antibody to SDF-1, an antibody to CXCR4, or a CXCR4 inhibitor.

10. The method of claim 9, wherein said CXCR4 inhibitor is T22[Tyr^{5,12}, Lys⁷]-polyphemusin II, ALX40-4C, or AMD3100.
